(19) United States
(12) Patent Application Publication
(10) Pub. No.: US 2022/0213031 A1
(43) Pub. Date: Jul. 7, 2022

(54) PROCESS FOR WORKING UP A COMPOSITION COMPRISING SOLID 4,4'-DICHLORODIPHENYL SULFOXIDE AND A SOLVENT

(71) Applicant: BASF SE, Ludwigshafen am Rhein (DE)

(72) Inventors: Indre THIEL, Ludwigshafen am Rhein (DE); Christian SCHUETZ, Ludwigshafen am Rhein (DE); Jessica Nadine HAMANN, Ludwigshafen am Rhein (DE); Stefan BLEI, Ludwigshafen am Rhein (DE)

(21) Appl. No.: 17/602,459

(22) PCT Filed: Apr. 3, 2020
(86) PCT No.: PCT/EP2020/059612

§ 371 Date: Oct. 8, 2021

(30) Foreign Application Priority Data
Apr. 9, 2019 (EP) 19168118.8

(51) Int. Cl. C07C 315/06

(52) U.S. Cl. CPC C07C 315/06

(57) ABSTRACT

The invention relates to a process for working up a composition comprising solid 4,4′-dichlorodiphenyl sulfoxide and a solvent, wherein the amount of solvent in the composition is in the range between 5 and 25 wt % based on the total mass of the composition by washing the composition using a carboxylic acid until the amount of solvent in the composition is below 1.5 wt % based on the total amount of the composition after washing.

4,4′-dichlorodiphenyl sulfoxide (in the following also termed as “DCDPSO”) is used as a precursor for producing 4,4′-dichlorodiphenyl sulfone which is used, for example, as a monomer for preparing polymers like polysulfone or polyether sulfone or as an intermediate of pharmaceuticals, dyes and pesticides.

For the production of DCDPSO several processes are known. One common process is a Friedel-Crafts reaction with thionyl chloride and chlorobenzene as starting materials in the presence of a catalyst, for example aluminum chloride. Generally the reaction of thionyl chloride and chlorobenzene is disclosed as a first part in the production of 4,4′-dichlorodiphenyl sulfone. For this purpose thionyl chloride and chlorobenzene are reacted in the presence of a catalyst. In a next step, the intermediate reaction product obtained by the reaction of thionyl chloride and chlorobenzene is hydrolyzed at an elevated temperature.

General processes for the production of sulfur containing diaryl compounds are disclosed, for example, in Sun, X. et al, “Investigations on the Lewis-acids-catalyzed electrophilic aromatic substitution reactions of thionyl chloride and selenyl chloride, the substituent effect, and the reaction mechanisms”, Journal of Chemical Research 2013, pages 736 to 744, Sun, X. et al, “Formation of diphenyl sulfoxide and diphenyl sulfide via the aluminum chloride-facilitated electrophilic aromatic substitution of benzene with thionyl chloride, and a novel reduction of sulfur(IV) to sulfur(II)”, Phosphorus, Sulfur, and Silicon, 2010, Vol. 185, pages 2535-2542 and Sun, X. et al., “Iron(II) chloride (FeCl₃)-catalyzed electrophilic aromatic substitution of chlorobenzene with thionyl chloride (SOCl₂) and the accompanying auto-redox in sulfur to give diaryl sulfides (Ar₂S): Comparison to catalysis by aluminum chloride (AlCl₃)”. In these papers different reaction conditions and catalysts are compared.

CN-A 108047101, for example, describes a Friedel-Crafts acylation reaction of thionyl chloride and chlorobenzene in the presence of a Lewis acid catalyst followed by a hydrolysis. The obtained reaction product is separated into an aqueous phase and an organic phase. The organic phase is subjected to an oxidization using hydrogen peroxide, acetic acid or sulfuric acid to obtain 4,4′-dichlorodiphenyl sulfone.

Further processes for producing 4,4′-dichlorodiphenyl sulfone are disclosed in CN-A 102351756, CN-A 102351757 and CN-A 102351758. In these patent applications, the first part of the process for producing DCDPSO is similar. In a first step a Friedel-Crafts reaction is carried out using thionyl chloride and chlorobenzene as raw materials and aluminum chloride as catalyst. After completion of the Friedel-Crafts reaction, the mother liquor is hydrolyzed followed by refluxing. In a next step the resulting mixture is cooled to allow separation into an aqueous phase and an organic phase. The organic phase is subjected to vacuum distillation, centrifugation and washing to obtain DCDPSO as product.

According to the examples of CN-A 104557626 the Friedel-Crafts reaction of thionyl chloride and chlorobenzene in the presence of aluminum chloride is carried out at 20° C., 25° C. and 30° C. However, this document does not mention whether additional steps are necessary to obtain the DCDPSO which is used for the further oxidation reaction to obtain 4,4′-dichlorodiphenyl sulfone.

A further process for producing 4,4′-dichlorodiphenyl sulfone in a two-stage process where in the first stage DCDPSO is produced is disclosed in CN-B 104402780. For producing DCDPSO, a Friedel-Crafts reaction is carried out using thionyl chloride and chlorobenzene as raw material and anhydrous aluminum chloride as catalyst. The Friedel-Crafts reaction is followed by cooling, hydrolysis, heating and refluxing. After reflux is finished, the reaction mixture is cooled down and DCDPSO precipitates in form of white crystals which are filtered off. The DCDPSO then is oxidized to obtain 4,4′-dichlorodiphenyl sulfone.

SU-A 765262 also discloses a process for producing 4,4′-dichlorodiphenyl sulfone in a two-stage process where in the first stage DCDPSO is obtained by a Friedel-Crafts reaction using thionyl chloride and chlorobenzene in the presence of aluminum chloride. According to the examples, the mixture obtained in the Friedel-Crafts reaction is poured into a 3% aqueous solution of hydrochloric acid and heated to completely dissolve the DCDPSO in the chlorobenzene which is added in excess. After separation into two phases, the organic phase is washed and then cooled to precipitate the DCDPSO.

It is an object of the present invention to provide a process for working up a composition comprising solid DCDPSO and a solvent giving DCDPSO in high quality which can be used for producing 4,4′-dichlorodiphenyl sulfone. In particular a process was aimed at which yields DCDPSO which in the manufacture of 4,4′-dichlorodphenyl sulfone does not give rise to or at least essentially avoids toxic by-products. Moreover a process yielding DCDPSO was aimed at which can be used in a process for making 4,4′-dichlorodiphenyl sulfone which dispenses with special explosion-proof equipment. Further a working-up process was sought which avails the solvent used in the production of DCDPSO for recycling.

This object is achieved by a process for working up a composition comprising solid DCDPSO and a solvent, wherein the amount of solvent in the composition is in the range between 5 and 25 wt % based on the total mass of the composition by washing the composition using a carboxylic acid until the amount of solvent in the composition is below 1.5 wt % based on the total amount of the composition after washing.

By washing the composition comprising solid DCDPSO and a solvent (in the following also termed as “composition”) with a carboxylic acid, the solvent in the composition is at least partly removed. The carboxylic acid used for washing preferably corresponds to the carboxylic acid used in a subsequent process for oxidizing the DCDPSO forming 4,4′-dichlorodiphenyl sulfone.

The solvent in the composition generally is a solvent which is used in a process for producing DCDPSO. Depending on the process for producing DCDPSO, the solvent particularly is chlorobenzene. In the context of the present invention the person skilled in the art appreciates that the term “chlorobenzene” means monochlorobenzene which may contain traces of impurities.

An amount of solvent, particularly chlorobenzene, below 1.5 wt %, more preferred below 1 wt % allows to use the obtained composition comprising DCDPSO and carboxylic acid (in the following also termed as “washed composition”) as such or DCDPSO isolated therefrom to produce 4,4′-dichlorodiphenyl sulfone without generating an explosive gas phase or liquid phase. Further, such a low amount of solvent reduces the formation of toxic by-product to an extent which has no detrimental effect on the further use of the 4,4′-dichlorodiphenyl sulfone produced by oxidation of the DCDPSO worked up according to the invention.

The composition comprises 5 to 25 wt % solvent, preferably 7 to 15 wt % solvent and particularly 8 to 12 wt % solvent, each based on the total mass of the composition. The composition which is worked up by the inventive process can be made by mixing DCDPSO with solvent. Usually the composition directly arises from a production process of DCDPSO, for example it is the residual moisture containing solid phase obtained in a solid-liquid-separation process of a suspension comprising solid DCDPSO and a solvent, for example a filtration or centrifugation. The amount of solvent remaining in the composition thereby depends on the filtration or centrifugation process. If the solid-liquid-separation process is a filtration, the residual moisture containing solid phase also is denoted as “filter cake”.

Washing of the composition can be carried out in any apparatus which allows washing of a residual moisture containing compound. An apparatus which can be used for the washing for example is a stirred tank or filtration apparatus. In a preferred embodiment, the apparatus used for washing is a filtration apparatus. Using a filtration apparatus has the advantage that a much smaller amount of carboxylic acid for washing is needed to achieve the required amount of solvent in the composition after washing. If a filtration apparatus is used for washing, the amount of carboxylic acid used for washing the composition preferably is at least 0.15 times the total mass of the composition, more preferred at least 0.2 times the total mass of the composition and particularly at least 0.5 times the total mass of the composition. The maximum amount of carboxylic acid used for washing the composition preferably is 3 times the total mass of the composition, more preferred 2 times the total mass of the composition and particularly 1.5 times the total mass of the composition if a filtration apparatus is used for washing the composition. If a stirred tank is used for washing, the amount of carboxylic acid for washing preferably is in the range between 0.5 and 3 times the total mass of the composition, more preferred 1 to 2 times the total mass of the composition and particularly 1 to 1.5 times the total mass of the composition.

If the washing is carried out in a stirred tank, a solid-liquid-separation can take place after washing the composition. For solid-liquid-separation any operation known to a skilled person can be used. Suitable solid-liquid-separation operations for example are filtration or centrifugation. If the solid-liquid-separation is a filtration, any filtration apparatus can be used.

It is particularly preferred that the composition is obtained in a filtration process and that the filtration and the subsequent washing of the composition are carried out in the same filtration apparatus. Suitable filtration apparatus are for example an agitated pressure strainer (pressure nutsche), a rotary pressure filter, a drum filter or a belt filter. The pore size of the filters used in the filtration apparatus preferably is in the range between 1 and 1000 μm, more preferred in the range between 10 and 500 μm and particularly in the range between 20 and 200 μm.

By means of the washing, solvent is replaced by carboxylic acid in the composition comprising DCDPSO. The washed composition comprises DCDPSO, carboxylic acid and remainders of solvent in an amount of less than 1.5 wt % based on the total amount of the composition, more preferred in an amount of less than 1.2 wt % based on the total amount of the composition and particularly in an amount of less than 1 wt % based on the total amount of the composition. The amount of carboxylic acid in the washed composition preferably is in a range between 6 and 30 wt % based on the total mass of the composition, more preferred in a range between 9 and 25 wt % based on the total mass of the composition and particularly in a range between 9 and 15 wt % based on the total mass of the composition. The wt % ranges given above refer to the washed composition after the filtration has been carried out in a filtration apparatus or, if the washing is carried out in a stirred tank, after the solid-liquid separation following the washing.

If the washing is carried out in a stirred tank, to remove the solvent, it is necessary to withdraw the whole liquid from the stirred tank after finishing the washing. To achieve the desired weight ratio of DCDPSO to carboxylic acid it can be necessary to add fresh carboxylic acid.

In the process for producing 4,4′-dichlorodiphenyl sulfone using the DCDPSO, generally a mixture is used comprising DCDPSO and carboxylic acid in a weight ratio of DCDPSO to carboxylic acid in a range from 1:2 to 1:6, more preferred in a range from 1:2 to 1:4 and particularly in a range from 1:2.5 to 1:3.5. To achieve this ratio, additional carboxylic acid can be added to the washed composition after washing. By such a ratio of DCDPSO to carboxylic acid, the solubility of 4,4′-dichlorodiphenyl sulfone produced by oxidation of the DCDPSO is at an optimum at the temperature of the oxidization reaction and a subsequent crystallization process for obtaining crystallized 4,4′-dichlorodiphenyl sulfone. Such a ratio particularly allows a sufficient heat dissipation in the reaction and an amount of 4,4′-dichlorodiphenyl sulfone in the mother liquor obtained by crystallization which is as low as possible.

During the washing a liquid mixture comprising solvent and carboxylic acid is obtained and withdrawn from the washing apparatus. To reduce the amount of carboxylic acid and solvent to be disposed, it is preferred to separate the liquid mixture comprising solvent and carboxylic acid into a first stream comprising substantially solvent and a second stream comprising substantially carboxylic acid. This allows the first and second streams to be recycled or used in different processes which use either carboxylic acid or solvent. “Comprising substantially solvent” in this context means that the first stream comprises preferably at least 95 wt % solvent, more preferred at least 98 wt % solvent and particularly at least 99 wt % solvent, each based on the total amount of the first stream. The second stream preferably comprises at least 80 wt % carboxylic acid, more preferred at least 85 wt % carboxylic acid and particularly at least 88 wt % carboxylic acid, each based on the total amount of the second stream. The reason for the lower content of carboxylic acid in the second stream compared to the amount of solvent in the first stream is that the liquid mixture still contains a considerable amount of DCDPSO. This can for instance be an amount of about 10 wt % based on the total amount of the first stream. As the DCDPSO is a high boiler compared to the solvent, the DCDPSO also collects in the second stream.

It is particularly preferred to recycle the second stream comprising substantially carboxylic acid into the washing of the composition. The first stream comprising substantially solvent preferably is recycled into a production process of DCDPSO.

Separation of the liquid mixture into the first and second streams can be obtained for example by distillation or evaporation. If the separation is carried out by distillation, usually a distillation column is used. The distillation column may have internals, for example trays, a structured packing or a random packing or a combination of at least two thereof. If the separation is carried out as an evaporation, any evaporator known to a skilled person can be used. Suitable evaporators for example are falling film evaporators, thin film evaporators or natural or forced circulation evaporators. Particularly preferred is evaporation in a falling film evaporator or distillation in a distillation column. Evaporation or distillation preferably is carried out at a pressure in the range from 20 to 700 mbar(abs), more preferred in the range from 50 to 500 mbar(abs) and particularly in a range from 70 to 200 mbar(abs) and a temperature in the range from 130 to 200° C., more preferred from 140 to 180° C. and particularly in a range from 150 to 170° C. in the bottom of the distillation column.

The carboxylic acid used for washing the composition can be only one carboxylic acid or a mixture of at least two different carboxylic acids. Preferably the carboxylic acid is at least one aliphatic carboxylic acid. The at least one aliphatic carboxylic acid may be at least one linear or at least one branched aliphatic carboxylic acid or it may be a mixture of one or more linear and one or more branched aliphatic carboxylic acids. Preferably the aliphatic carboxylic acid is an aliphatic C₆ to C₁₀ carboxylic acid, particularly a C₆ to C₉ carboxylic acid, whereby it is particularly preferred that the at least one carboxylic acid is an aliphatic monocarboxylic acid. Thus, the at least one carboxylic acid may be hexanoic acid, heptanoic acid, octanoic acid nonanoic acid or decanoic acid or a mixture of one or more of said acids. For instance, the at least one carboxylic acid may be n-hexanoic acid, 2-methyl-pentanoic acid, 3-methyl-pentanoic acid, 4-methylpentanoic acid, n-heptanoic acid, 2-methyl-hexanoic acid, 3-methyl-hexanoic acid, 4-methylhexanoic acid, 5-methyl-hexanoic acid, 2-ethyl-pentanoic acid, 3-ethyl-pentanoic acid, n-octanoic acid, 2-methyl-heptanoic acid, 3-methyl-heptanoic acid, 4-methyl-heptanoic acid, 5-methyl-heptanoic acid, 6-methyl-heptanoic acid, 2-ethyl-hexanoic acid, 4-ethyl-hexanoic acid, 2-propyl pentanoic acid, 2,5-dimethylhexanoic acid, 5,5-dimethyl-hexanoic acid, n-nonanoic acid, 2-ethyl-hepatnoic acid, n-decanoic acid, 2-ethyl-octanoic acid, 3-ethyl-ocantoic acid, 4-ethyl-octanoic acid. The carboxylic acid may also be a mixture of different structural isomers of one of said acids. For instance, the at least one carboxylic acid may be isononanoic acid comprising a mixture of 3,3,5-trimethyl-hexanoic acid, 2,5,5-trimethyl-hexanoic acid and 7-methyloctanoic acid or neodecanoic acid comprising a mixture of 7,7-dimethyloctanoic acid, 2,2,3,5-tetramethyl-hexanoic acid, 2,4-dimethyl-2-isopropylpentanoic acid and 2,5-dimethyl-2-ethylhexanoic acid. Particularly preferably, however the carboxylic acid is hexanoic acid or heptanoic acid.

The composition comprising solid DCDPSO and a solvent preferably is obtained by a process for producing DCDPSO comprising:
(I) reacting thionyl chloride, chlorobenzene and aluminum chloride in a molar ratio of thionyl chloride:chlorobenzene:aluminum chloride of 1:(6 to 9):(1 to 1.5) at a temperature in the range from 0 to below 20° C., forming an intermediate reaction product and hydrogen chloride;
(II) mixing aqueous hydrochloric acid and the intermediate reaction product at a temperature in the range from 70 to 110° C. to obtain an organic phase comprising DCDPSO and an aqueous phase;
(III) cooling the organic phase comprising the DCDPSO to a temperature below the saturation point of DCDPSO to obtain a suspension comprising crystallized DCDPSO;
(IV) solid-liquid-separation of the suspension to obtain a residual moisture containing solid DCDPSO, wherein the residual moisture containing solid DCDPSO comprises crystallized DCDPSO and mother liquor.

Thereafter the DCDPSO can be collected and used as composition to be worked up according to the process as disclosed herein. Alternatively the DCDPSO can be washed with solvent (solvent-washing) and thus further purified. The thus further purified DCDPSO can be collected and used as the composition which is worked-up by washing with carboxylic acid.

To obtain DCDPSO, in the reaction (I) thionyl chloride, chlorobenzene and aluminum chloride are fed into a reactor in a molar ratio of thionyl chloride:chlorobenzene:aluminum chloride of 1:(6 to 9):(1 to 1.5), preferably in a molar ratio of thionyl chloride:chlorobenzene:aluminum chloride of 1:(7 to 9):(1 to 1.2) and particularly in a molar ratio of thionyl chloride:chlorobenzene:aluminum chloride of 1:(7 to 8):(1 to 1.1).

The reactor can be any reactor which allows mixing and reacting of the components fed into the reactor. A suitable reactor is for example a stirred tank reactor or jet loop reactor. The reaction can be operated either continuously or batchwise. Preferably, the reaction is operated batchwise.

The thionyl chloride, chlorobenzene and aluminum chloride can be added simultaneously or successively. For reasons of ease of conduct of the reaction—in particular in case of batch reaction—preferably, aluminum chloride and chlorobenzene are fed firstly into the reactor and then the thionyl chloride is added to the aluminum chloride and chlorobenzene. In this case the aluminum chloride and chlorobenzene can be added simultaneously or one after the other. However, in each case it is preferred to mix the aluminum chloride and chlorobenzene before adding the thionyl chloride. During the reaction hydrogen chloride (HCl)—typically in gaseous form—is formed which is at least partially withdrawn from the reactor. The volumetric flow for adding the thionyl chloride typically depends on heat dissipation and flow rate of the gas withdrawn from the reactor.

The chlorobenzene which is added in excess into the reactor and, therefore, only partially converted during the chemical reaction, also serves as a solvent for the reaction products. In any step of the process in which a solvent is used, the solvent preferably is chlorobenzene.

The thionyl chloride and the chlorobenzene react in the presence of the aluminum chloride whereby an intermediate reaction product and hydrogen chloride form. The intermediate reaction product comprises 4,4′-dichlorodiphenyl sulfoxide-AlCl₃ adduct. The aluminum chloride generally can act as catalyst. The chemical reaction can be schematically represented by the following chemical reaction equation (1):

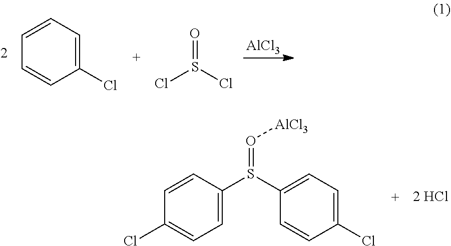

The reaction can be carried out at a constant or almost constant temperature. It is also possible to carry out the reaction at varying temperatures within the described range, for instance employing a temperature profile over the time of reaction or the reactor.

Independently of whether the reaction is operated continuously or batchwise, the flow rate of the thionyl chloride is selected such that the heat generated by the reaction can be dissipated from the reactor by suitable cooling devices to keep the temperature in the reactor within a predefined range.

The hydrogen chloride (HCl) produced in the reaction typically is in gaseous form and at least partly removed from the reactor. While it can be put to other use in gaseous form, preferably, the hydrogen chloride removed from the reaction is mixed with water to produce aqueous hydrochloric acid.

After the reaction the intermediate reaction product is mixed with aqueous hydrochloric acid. For reasons of energy as well as production efficiency as well as sustainability, particularly preferably, the aqueous hydrochloric acid is produced from the hydrogen chloride removed from the reaction (I). By mixing the intermediate reaction product with the aqueous hydrochloric acid hydrolysis of the intermediate reaction product can take place. A crude reaction product comprising DCDPSO is obtained. The crude reaction product can also comprise aluminum chloride which is typically in hydrated form, usually as AlCl₃.6H₂O. The hydrolysis can be schematically represented by reaction equation (2):

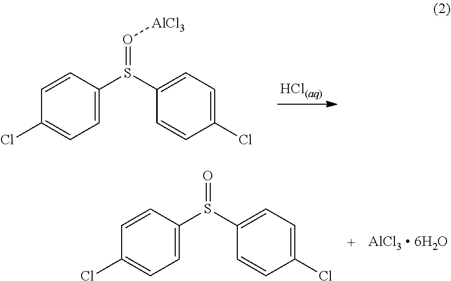

To facilitate the hydrolysis and to bring it as fast as possible to completion, the mixture can be agitated, preferably the mixture is stirred. After finishing the hydrolysis, the mixture separates into an aqueous phase comprising the AlCl₃ and an organic phase comprising DCDPSO solved in the excess chlorobenzene. In case the mixture is stirred, stirring is stopped to allow the mixture to separate.

The aqueous hydrochloric acid may have any concentration. However, a concentration of the hydrochloric acid above 3 wt % improves the solubility of the aluminum chloride. Preferably, the aqueous hydrochloric acid used in the hydrolysis has a concentration in the range from 3 to 12 wt %. All concentrations of hydrochloric acid in wt % above and in the following are based on the total amount of hydrogen chloride and water in the aqueous hydrochloric acid. An advantage of a higher concentration, particularly of a concentration in the range from 10 to 12 wt %, is that the density of the aqueous phase increases and the aqueous phase thus forms the lower phase whereas the upper phase is the organic phase comprising the DCDPSO, in the following also termed as “organic phase”. This allows an easier draining of the aqueous phase to obtain the organic phase.

The amount of aqueous hydrochloric acid used in (II) preferably is such that no aluminum chloride precipitates and that further two liquid phases are formed, the lower phase being the aqueous phase and the organic phase being the upper phase. To achieve this, the amount of aqueous hydrochloric acid added to the reaction mixture preferably is such that after the hydrolysis the weight ratio of aqueous to organic phase is in the range from 0.6 to 1.5 kg/kg. A smaller amount of aqueous hydrochloric acid may result in precipitation of aluminum chloride. Particularly at higher concentrations of the aqueous hydrochloric acid, a larger amount is necessary to avoid precipitation. Therefore, the concentration of the aqueous hydrochloric acid preferably is kept below 12 wt %.

The reaction of thionyl chloride, chlorobenzene and aluminum chloride and the mixing with aqueous hydrochloric acid and thus the hydrolysis can be carried out in the same reactor or in different reactors. Preferably, the reaction is carried out in a first reactor and the hydrolysis in a second reactor. If a first reactor and a second reactor are used, the first reactor corresponds to the reactor as described above. The second reactor also can be any reactor to perform a batchwise reaction and which allows stirring of the components in the reactor. Therefore, the second reactor also preferably is a stirred tank reactor.

Either the one reactor, if the reaction and the hydrolysis are carried out in the same reactor, or the preferably used first and second reactors is, respectively are designed in such a way that the temperature can be set to adjust the temperature in the reactor. For this purpose it is, for example, possible to provide a pipe inside the reactor through which a heating medium or a cooling medium can flow.

If the reaction and the hydrolysis are carried out in different reactors, it is particularly preferred to heat the intermediate reaction product to a temperature which is above the solubility point of the intermediate reaction product in the solvent after the reaction is completed and prior to transporting the intermediate reaction product from the first reactor to the second reactor. The solubility point denotes the temperature of the reaction mixture at which the intermediate reaction product is fully dissolved in the solvent.

If the reaction and the hydrolysis are carried out in the same reactor, the aqueous hydrochloric acid is fed into the reactor after the reaction is completed and after the intermediate reaction product is heated to the temperature of the hydrolysis. The flow rate of the aqueous hydrochloric acid preferably is set such that the temperature of the hydrolysis can be held in the specified range for the hydrolysis by tempering the reactor. If the reaction and the hydrolysis are carried out in different reactors, it is preferred to firstly feed the aqueous hydrochloric acid into the second reactor and to add the intermediate reaction product to the aqueous hydrochloric acid. In this case the flow rate of adding the intermediate reaction product into the second reactor is set such that the temperature in the second reactor is held within the specified temperature limits for the hydrolysis by tempering the second reactor.

To remove the aqueous hydrochloric acid and remainders of the aluminum chloride from the organic phase, the organic phase obtained in (II) preferably is separated off and washed with an extraction liquid before cooling in (III).

The phase separation following the hydrolysis can be carried out in the reactor in which the hydrolysis took place or in a separate vessel for phase separation. Under the aspect of less complexity, preferably the phase separation is carried out in the reactor in which the hydrolysis took place. After the phase separation is completed, the aqueous phase and the organic phase are removed separately from the vessel in which the phase separation took place, preferably the reactor in which the hydrolysis was performed.

After being separated off, the organic phase is washed with an extraction liquid to remove residual aluminum chloride and hydrochloric acid. The extraction liquid used for washing the organic phase preferably is water. Particularly preferably, the water which is used for washing the organic phase is separated off after washing and mixed with the hydrogen chloride obtained in (I) to obtain the aqueous hydrochloric acid.

Washing with the extraction liquid preferably is carried out in a separate washing vessel. However, it is also possible to only remove the aqueous phase from the reactor in which the hydrolysis took place and carry out the washing step in the reactor in which the hydrolysis took place. If the washing is carried out in a separate washing vessel, any vessel in which an organic phase can be washed can be used. The washing vessel usually comprises means to intimately mix the organic phase with the extraction liquid. Preferably, the washing vessel is a stirred tank into which the organic phase and the extraction liquid are fed and then mixed.

If the phase separation is carried out in a vessel for phase separation, the washing with the extraction liquid either can be carried out in a washing vessel or, alternatively, in the vessel for phase separation. If phase separation and washing are carried out in the same vessel, it is necessary to provide means for mixing the organic phase with the extraction liquid after the aqueous phase, which was separated from the organic phase, is drained off.

The washing with the extraction liquid preferably is carried out at the same temperature as the hydrolysis.

Generally, the amount of extraction liquid which preferably is water is sufficient to remove all or essentially all of the aluminum chloride from the organic phase. Under the aspect of waste control it is usually preferred to use as little extraction liquid as possible. If water is used as extraction liquid, it is particularly preferred to use such an amount of water that the entire aqueous phase from the washing step can be used to generate the aqueous hydrochloric acid in the concentration needed for hydrolysis. For this purpose, the water which is used for washing is separated off and mixed with the hydrogen chloride obtained in the reaction to obtain the aqueous hydrochloric acid.

After a predetermined period for washing with the extraction liquid, mixing is stopped to allow the mixture to separate into an aqueous phase and an organic phase. The aqueous phase and the organic phase are removed from the washing vessel separately.

For separating the DCDPSO from the organic phase, the organic phase is cooled to a temperature below the saturation point of DCDPSO in (III) to obtain a suspension comprising crystallized DCDPSO (in the following also termed as “suspension”).

The saturation point denotes the temperature of the organic phase at which DCDPSO starts to crystallize. This temperature depends on the concentration of the DCDPSO in the organic phase. The lower the concentration of DCDPSO in the organic phase, the lower is the temperature at which crystallization starts.

The cooling (Ill) for crystallizing DCDPSO can be carried out in any crystallization apparatus or any other apparatus which allows cooling of the organic phase, for example an apparatus with surfaces that can be cooled, such as a vessel or a tank with cooling jacket, cooling coils or cooled baffles like so called “power baffles”.

Cooling of the organic phase for crystallization of the DCDPSO can be performed either continuously or batchwise. To avoid precipitation and fouling on cooled surfaces, it is preferred to carry out the cooling in a gastight closed vessel by

(i) reducing the pressure in the gastight closed vessel;

(ii) evaporating solvent;

(iii) condensing the evaporated solvent by cooling;

(iv) returning the condensed solvent into the gastight closed vessel.

This process allows for cooling the organic phase without cooled surfaces onto which crystallized DCDPSO accumulates and forms a solid layer. This enhances the efficiency of the cooling process. Also, additional efforts to remove this solid layer can be avoided. Therefore, it is particularly preferred to use a gastight closed vessel without cooled surfaces.

To avoid precipitation of the crystallized DCDPSO it is further preferred to agitate the organic phase in the crystallization apparatus. Therefore, a suitable apparatus is, for example, a stirred tank or a draft-tube crystallizer.

To crystallize DCDPSO, it is necessary to provide crystal nuclei. To provide the crystal nuclei it is possible to use dried crystals, which are added to the organic phase, or to add a suspension comprising particulate DCDPSO as crystal nuclei. If dried crystals are used but the crystals are too big, it is possible to grind the crystals into smaller particles which can be used as crystal nuclei. Further, it is also possible to provide the necessary crystal nuclei by applying ultrasound to the organic phase. Preferably, the crystal nuclei are generated in situ in an initializing step. The initializing step preferably comprises following steps before setting the reduced pressure in step (i):
reducing the pressure in the gastight closed vessel such that the boiling point of the organic phase is in the range from 80 to 95° C.;
evaporating solvent until an initial formation of solids takes place;
increasing the pressure in the vessel and heating the organic phase in the vessel to a temperature in the range from 85 to 100° C.

By reducing the pressure in the vessel such that the boiling point of the organic phase is in the range from 80 to 95° C., the following evaporation of solvent leads to a saturated solution and the precipitation of DCDPSO. By the following pressure increase and heating the organic phase in the gastight closed vessel to a temperature in the range from 85 to 100° C., the solidified DCDPSO starts to partially dissolve again. This has the effect that the number of crystal nuclei is reduced which allows producing a smaller amount of crystals with a bigger size. Cooling, particularly by reducing the pressure, can be started immediately after a pre-set temperature within the above ranges is reached to avoid complete dissolving of the produced crystal nuclei. However, it is also possible to start cooling after a dwell time of, for example, 0.5 to 1.5 h at the preset temperature.

For generating the crystal nuclei in the initializing step, it is possible to only evaporate solvent until an initial formation of solids take place. It is also possible to entirely condense the evaporated solvent by cooling and to return all the condensed solvent into the gastight closed vessel. The latter has the effect that the liquid in the gastight closed vessel is cooled and solid forms. A mixture of both approaches, where only a part of the evaporated and condensed solvent is returned into the gas tight vessel, is also viable.

If the cooling and thus the crystallization of DCDPSO is performed batchwise, it is preferred to carry out steps (ii) to (iv) during the pressure reduction in step (i). Thereby, it is particularly preferred to continuously reduce the pressure in step (i) until the temperature in the gastight closed vessel reaches a predefined value in the range from 0 to 45° C. After the predefined temperature value is reached, pressure reduction is stopped and then the gastight closed vessel is vented until ambient pressure is reached. The temperature profile in the gastight closed vessel preferably is selected such that the organic phase is subjected to a constant supersaturation.

To reduce the solubility of the DCDPSO and thus increase the yield of solidified DCDPSO it is necessary to shift the saturation point. This is possible by continuously reducing the amount of solvent at a constant temperature, for example by evaporating solvent, or by cooling the organic phase at constant concentration. Since reduction of the amount of solvent results in a very viscous suspension when a certain critical concentration is reached, it is preferred to increase the yield of solidified DCDPSO partly by reducing the amount of solvent by evaporation followed by reducing the temperature. For reducing the solubility of DCDPSO in the organic phase and to improve the crystallization, it is possible to additionally add at least one drowning-out agent, for example at least one protic solvent like water, an alcohol, and/or an acid, particularly a carboxylic acid, or at least one highly unpolar solvent like a linear and/or cyclic alkane. With respect to ease of workup water, methanol, ethanol, acetic acid and/or formic acid, particularly water and/or methanol are preferred drowning-out agents.

After reaching ambient pressure the suspension which formed in the gastight closed vessel by the cooling is withdrawn and fed into the solid-liquid-separation (IV).

If the cooling and thus the crystallization of DCDPSO is performed continuously, it is preferred to operate the cooling and crystallization stepwise in at least two steps, particularly in two to three steps. If the cooling and crystallization is carried out in two steps, in a first step the organic phase preferably is cooled to a temperature in the range from 40 to 90° C. and in a second step preferably to a temperature in the range from −10 to 50° C. If the cooling is operated in more than two steps, the first step preferably is operated at a temperature in the range from 40 to 90° C. and the last step at a temperature in the range from −10 to 30° C. The additional steps are operated at temperatures between these ranges with decreasing temperature from step to step.

As in the batchwise process, the temperature in the continuously operated process can be set by using apparatus for cooling and crystallization having surfaces to be cooled, for example a cooled jacket, cooling coils or cooled baffles like so called “power baffles”. To establish the at least two steps for cooling and crystallization, for each step at least one apparatus for cooling and crystallization is used. To avoid precipitation of DCDPSO, also in the continuous process it is preferred to reduce the temperature by reducing the pressure in the apparatus for cooling and crystallization wherein the apparatus for cooling and crystallization preferably are gastight closed vessels. Suitable apparatus for cooling and crystallization for example are agitated-tank crystallizers, draft-tube crystallizers, horizontal crystallizers, forced-circulation crystallizers or Oslo-crystallizers. The pressure which is set to achieve the required temperature corresponds to the vapor pressure of the organic phase. Due to the pressure reduction, low boilers, particularly solvent, evaporate. The evaporated low boilers are cooled to condense, and the condensed low boilers are returned into the respective apparatus for cooling and crystallization by which the temperature is set.

If the cooling and crystallization is carried out continuously, a stream of the suspension is continuously withdrawn from the apparatus for cooling and crystallization. The suspension then is fed into the solid-liquid-separation (IV). To keep the liquid level in the apparatus for cooling and crystallization within predefined limits, fresh organic phase can be fed into the apparatus in an amount corresponding or essentially corresponding to the amount of suspension withdrawn from the apparatus. The fresh organic phase either can be added continuously or batchwise each time a minimum liquid level in the apparatus for cooling and crystallization is reached. Generally, the process can comprise that hydrolysis (II) is carried out batchwise or continuously and that cooling is carried out batchwise or continuously. Thus, it can comprise that hydrolysis (II) is carried out batchwise and cooling continuously or vice versa. If the hydrolysis in (II) is carried out batchwise and the organic phase shall be added continuously into the apparatus for cooling and crystallization or must be added at times when the hydrolysis is not yet finished or if the hydrolysis is operated continuously and the cooling batchwise, preferably at least one buffer container is used into which the organic phase is fed after being withdrawn from the hydrolysis. From this buffer container the organic phase then is fed into the apparatus for cooling and crystallization.

Independently of whether it is carried out batchwise or continuously, crystallization preferably is continued until the solids content in the suspension in the last step of the crystallization is in the range from 5 to 50 wt %, more preferred in the range from 5 to 40 wt % and particularly in the range from 20 to 40 wt %, based on the mass of the suspension.

Independently of whether the cooling and crystallization is performed continuously or batchwise, the solid-liquid-separation (IV) can be carried out either continuously or batchwise, preferably continuously.

If the cooling and crystallization (III) is carried out batchwise and the solid-liquid-separation (IV) is carried out continuously, at least one buffer container is used into which the suspension withdrawn from the apparatus used for cooling and crystallization is filled. For providing the suspension a continuous stream is withdrawn from the at least one buffer container and fed into a solid-liquid-separation apparatus. The volume of the at least one buffer container preferably is such that each buffer container is not totally emptied between two filling cycles in which the contents of the apparatus for cooling and crystallization is fed into the buffer container. If more than one buffer container is used, it is possible to fill one buffer container while the contents of another buffer container are withdrawn and fed into the solid-liquid-separation. In this case the at least two buffer containers are connected in parallel. The parallel connection of buffer containers further allows filling the suspension into a further buffer container after one buffer container is filled. An advantage of using at least two buffer containers is that the buffer containers may have a smaller volume than only one buffer container. This smaller volume allows a more efficient mixing of the suspension to avoid sedimentation of the crystallized DCDPSO. To keep the suspension stable and to avoid sedimentation of solid DCDPSO in the buffer container, it is possible to provide the buffer container with a device for agitating the suspension, for example a stirrer, and to agitate the suspension in the buffer container.

If the cooling and crystallization (III) and the solid-liquid-separation (IV) are carried out batchwise, the contents of the vessel for cooling and crystallization directly can be fed into a solid-liquid-separation apparatus as long as the solid-liquid separation apparatus is large enough to take up the whole contents of the vessel for cooling and crystallization. In this case it is possible to omit the buffer container. It is also possible to omit the buffer container when cooling and crystallization and the solid-liquid-separation are carried out continuously. In this case also the suspension directly is fed into the solid-liquid-separation apparatus. If the solid-liquid separation apparatus is too small to take up the whole contents of the vessel for cooling and crystallization, also for batchwise operation at least one additional buffer container is necessary to allow to empty the crystallization apparatus and to start a new batch.

If the cooling and crystallization (III) are carried out continuously and the solid-liquid-separation (IV) is carried out batchwise, the suspension withdrawn from the cooling and crystallization apparatus is fed into the buffer container and each batch for the solid-liquid-separation is withdrawn from the buffer container and fed into the solid-liquid-separation apparatus.

The solid-liquid-separation (IV) for example comprises a filtration, centrifugation or sedimentation. Preferably, the solid-liquid-separation is a filtration. In the solid-liquid-separation liquid mother liquor is removed from the solid DCDPSO and residual moisture containing DCDPSO (in the following also termed as “moist DCDPSO”) is obtained. If the solid-liquid-separation (IV) is a filtration, the moist DCDPSO is called “filter cake”.

To carry out the solid-liquid-separation (IV) any solid-liquid-separation apparatus known by the skilled person can be used. Suitable solid-liquid-separation apparatus are, for example, an agitated pressure nutsche, a rotary pressure filter, a drum filter, a belt filter or a centrifuge. The pore size of the filters used in the solid-liquid-separation apparatus preferably is in the range from 1 to 1000 μm, more preferred in the range from 10 to 500 μm and particularly in the range from 20 to 200 μm.

Particularly preferably, cooling and crystallization (III) is carried out batchwise and the solid-liquid-separation (IV) is operated continuously.

As by cooling the majority of DCDPSO crystallizes but still a considerable amount of the DCDPSO remains dissolved in the solvent, the mother liquor withdrawn from the solid-liquid-separation apparatus preferably is concentrated and at least a part of the concentrated mother liquor is recycled into the cooling step (III). Concentration of the mother liquor preferably is performed by distillation or evaporation, preferably by evaporation. By concentrating the mother liquor and recycling the mother liquor into the cooling step (III) it is possible to reduce product loss to a minimum.

Evaporation or distillation preferably is continued until the concentration of DCDPSO in the mother liquor is in the range from 6 to 60 wt %, more preferred in the range from 10 to 50 wt %, and particularly in the range from 15 to 40 wt %, based on the total amount of concentrated mother liquor.

At least a part of the concentrated mother liquor is recycled into the cooling step (III). To avoid an excessive accumulation of high boiling byproducts and contaminants, it is preferred to recycle a part of the concentrated mother liquor into the cooling step (III) and to withdraw the rest of the concentrated mother liquor from the process. The amount of concentrated mother liquor recycled into the cooling step (III) preferably is in the range from 10 to 95 wt %, more preferred in the range from 40 to 90 wt %, and particularly in the range from 65 to 90 wt %, each based on the total amount of concentrated mother liquor.

The recycled concentrated mother liquor preferably is mixed with fresh organic phase and fed into the cooling (III). The ratio of fresh organic phase to concentrated mother liquor preferably is in the range from 60:1 to 6:1, more preferred in the range from 15:1 to 7:1 and particularly in the range from 10:1 to 7:1. The amount of concentrated mother liquor recycled into the cooling (III) preferably is set such that the amount of isomers of DCDPSO, particularly the amount of 2,4-dichlorodiphenyl sulfoxide, totally fed into the cooling (III) is in the range from 0 to 40 wt % and particularly in the range from 10 to 30 wt % based on the total amount of liquid fed into the cooling (III). The total amount of liquid fed into the cooling (III) is the sum of the organic phase containing DCDPSO obtained by mixing aqueous hydrochloric acid and intermediate product (II) and the recycled concentrated mother liquor. If the amount of isomers in the concentrated mother liquor rises, the part recycled into the cooling (III) is advantageously reduced, whereas a smaller amount of isomers in the concentrated mother liquor allows a larger part to be recycled, as long as the amount of isomers in the organic phase obtained by mixing aqueous hydrochloric acid and intermediate product (II) remains constant.

Mixing of the recycled concentrated mother liquor and the fresh organic phase can be carried out before feeding into the apparatus in which the cooling and crystallization takes place, such that a mixture of recycled concentrated mother liquor and fresh organic phase is fed into the apparatus. Alternatively, the recycled concentrated mother liquor and the fresh organic phase are fed separately into the apparatus in which the cooling and crystallization takes place and are mixed in this apparatus.

By concentrating and recycling at least a part of the mother liquor, the yield of DCDPSO can usually be increased considerably, such as up to about 10%, typically an increase of at least about 8 or 9%. This allows for carrying out the crystallization in only one step.

The moist DCDPSO obtained in the solid-liquid-separation (IV) still may contain impurities. To remove these impurities, an additional washing step with a washing liquid can be carried out to remove these impurities. By this additional washing with washing liquid, in the following also termed as “solvent-washing”, impurities are removed which may attach to the surface of the crystallized DCDPSO and which cannot be removed or cannot be sufficiently removed by washing with the carboxylic acid. Using the solvent for washing the moist DCDPSO in the solvent-washing has the additional advantage that impurities adhering to the surface of the crystallized DCDPSO can be removed because the DCDPSO starts to solve at the surface and thus the impurities adhering to the surface loosen and can be removed.

By this solvent-washing, the composition comprising solid DCDPSO and a solvent is obtained which then is washed with the carboxylic acid to remove the solvent by replacing the solvent by the carboxylic acid.

If the solid-liquid-separation is a filtration, it is possible to carry out the solvent-washing of the filter cake in the filtration apparatus independently of whether the filtration is operated continuously or batchwise. After solvent-washing, the filter cake is removed as the composition comprising solid DCDPSO and a solvent.

In a continuous solid-liquid-separation process, the moist DCDPSO can be removed continuously from the solid-liquid-separation apparatus and afterwards the solvent-washing of the moist DCDPSO takes place. In the case the solid-liquid separation (IV) is a filtration and a continuous belt filter is used, it is preferred to filtrate the suspension, to transport the thus originating filter cake on the filter belt and to wash the filter cake at a different position in the same filtration apparatus with the washing liquid.

If the solid-liquid separation (IV) is a filtration process, it is further also possible to operate the filtration semi-continuously. In this case the suspension is fed continuously into the filtration apparatus and the filtration is performed for a specified process time. Afterwards the filter cake produced during the filtration is washed with the washing liquid in the same filtration apparatus. The process time for performing the filtration for example may depend on the differential pressure. Due to the increasing filter cake the differential pressure in the filtration apparatus increases. To determine the process time for the filtration, it is for example possible to define a target differential pressure up to which the filtration is carried out in a first filtration apparatus. Thereafter the suspension is fed into a second or further filtration apparatus in which filtration is continued. This allows to continuously performing the filtration. In those apparatus where the filtration is completed, the filter cake can be washed with the washing liquid and withdrawn after finishing the solvent-washing. If necessary, the filtration apparatus can be cleaned after the filter cake is withdrawn. After the filter cake is withdrawn and the filter apparatus is cleaned when necessary, the filtration apparatus can be used again for filtration. If the washing of the filter cake and the optional cleaning of the filtration apparatus needs more time than the time for the filtration in one filtration apparatus, at least two filtration apparatus are used to allow continuous feeding of the suspension in a filtration apparatus while in the other apparatus the filter cake is washed with the washing liquid or the filtration apparatus are cleaned.

In each filtration apparatus of the semi-continuous process, the filtration is carried out batchwise. Therefore, if the filtration and solvent-washing are carried out batchwise, the process corresponds to the process in one apparatus of the above described semi-continuous process.

To reduce the amount of solvent used in the process, preferably at least a part of the solvent is purified after being used for washing the moist DCDPSO and recycled. The purification of the solvent can be carried out by each process known by a person skilled in the art. Particularly suitable are distillation or evaporation processes to separate impurities from the solvent. In the process, impurities which are washed out of the moist DCDPSO in the solvent-washing particularly are remainders of by-products, isomers of the DCDPSO and auxiliaries like catalysts used for the production of the DCDPSO. As these impurities which are washed out of the moist DCDPSO usually are higher boiling than the solvent, the purification of the solvent can be carried out by evaporation in which the solvent is evaporated and condensed in a subsequent condenser. In a distillation process, the solvent is removed from the distillation apparatus, preferably a distillation column, as top stream, and the bottom stream withdrawn from the distillation column contains the impurities. If the bottom stream still contains DCDPSO, it is also possible to recycle a part of the bottom stream into the cooling (III) to improve the yield and to reduce the amount of DCDPSO which is withdrawn from the process.

The thus purified solvent, for example, can be reused for washing the moist DCDPSO. Alternatively, it is also possible to recycle at least a part of the purified solvent into step (I).

If the solid-liquid-separation (IV) is carried out by centrifugation, depending on the centrifuge it might be necessary to use a separate washing apparatus for washing the moist DCDPSO. However, usually a centrifuge can be used which comprises a separation zone and a solvent-washing zone or the washing can be carried out after centrifuging in the centrifuge.

To avoid dissolving the DCDPSO in the solvent during the solvent-washing, it is preferred to keep the washing temperature at a temperature where the solubility of DCDPSO in the solvent is very low, preferably from 0 to 5 wt % based on the sum of DCDPSO and solvent.

If the solvent-washing is carried out in the filtration apparatus, the filter cake obtained after washing is the composition comprising DCDPSO which is washed with carboxylic acid. If the solvent-washing is carried out in a separate washing apparatus, an additional solid-liquid-separation may be necessary depending on the amount of solvent in the washed 4,4′-comprising composition obtained in the washing process.

The solvent used for washing the residual moisture containing solid DCDPSO comprising crystallized DCDPSO and mother liquor preferably is chlorobenzene, particularly monochlorobenzene. Therefore, the solvent in the composition comprising solid DCDPSO and a solvent which is washed with the carboxylic acid preferably is chlorobenzene and particularly monochlorobenzene.

Each process step described above can be carried out in only one apparatus or in more than one apparatus depending on the apparatus size and the amounts of compounds to be added. If more than one apparatus is used for a process step, the apparatus can be operated simultaneously or—particularly in a batchwise operated process—at different time. This allows for example to carry out a process step in one apparatus while at the same time another apparatus for the same process step is maintained, for example cleaned. Further, in those process steps where the contents of the apparatus remain for a certain time after all components are added, for example the reaction or the hydrolysis, after feeding all compounds in one apparatus it is possible to feed the components into a further apparatus while the process in the first apparatus still continues. However, it is also possible to add the components into all apparatus simultaneously and to carry out the process steps in the apparatus also simultaneously.

Due to the corrosivity of the components used in the process, it is preferred to provide all surfaces which come into contact with the components, particularly surfaces of the at least one reactor in which the reaction and the hydrolysis are carried out, the surfaces of the cooling vessel and each washing apparatus, with an enamel layer. Pipes connecting the apparatuses preferably are made of stainless steel with an enamel layer. The apparatus for each solid-liquid separation, particularly filtration apparatus, preferably is made of a nickel-base alloy or stainless steel with a corrosion resistant layer. If the solid-liquid-separation is a filtration, the filtration apparatus preferably comprises a filter element which is made of a material which has a good or very good chemical resistance. Such materials can be polymeric materials or chemical resistant metals as described above for the used apparatus. Filter elements for example can be filter cartridges, filter membranes, or filter cloth. If the filter element is a filter cloth, preferred materials additionally are flexible, particularly flexible polymeric materials such as those which can be fabricated into wovens. These can for instance be polymers which can be drawn or spun into fibers. Particularly preferred as material for the filter element are polyether ether ketone (PEEK), polyamide (PA) or fluorinated polyalkylenes, for example ethylene chlorotrifluoroethylene (ECTFE), polytetrafluoroethylene (PTFE), polyvinylidene fluoride (PVDF), fluorinated ethylenepropylene (FEP).

### EXAMPLES

Effect of Amount of Carboxylic Acid

A suspension comprising 84.7 wt % DCDPSO and 15 wt % monochlorobenzene (MCB) and the balance impurities like isomers of 4,4′-dichlorodiphenyl sulfoxide and further by-products of a production process of DCDPSO were subjected to a washing with heptanoic acid. Table 1 compiles the results regarding the composition of the filter cake depending on the conditions for replacing the MCB by heptanoic acid (HeptA). The amounts in wt % all are based on the total amount of the respective wet filter cake.

TABLE 1
Washing of MCB wet filter cake with HeptA

| | | Filter Cake Composition | | |
|---|---|---|---|---|
| | Ratio [g/g] | | | 4,4′- |
| 4,4′-DCDPSO Filter | Filter | MCB | HeptA | DCDPSO |
| Cake | cake:HeptA | [wt %] | [wt %] | [wt %] |
| MCB wet | | 15.0 | — | 84.7 |
| Washed with HeptA 1x | 1:1 (25° C.) | 0.3 | 16.5 | 82.9 |
| Washed with HeptA 2x | 1:1 (25° C.) | — | 17.2 | 82.4 |
| MCB wet | | 15.0 | — | 84.7 |
| Washed with HeptA 1x | 2:1 (25° C.) | 0.8 | 13.0 | 85.8 |
| Washed with HeptA 2x | 2:1 (25° C.) | 0.1 | 15.0 | 84.4 |
| MCB wet | | 15.0 | — | 84.7 |
| Washed with HeptA 1x | 2:1 (10° C.) | — | 13.7 | 81.9 |
| Washed with HeptA 2x | 2:1 (10° C.) | — | 13.3 | 82.9 |
| MCB wet | | 15.0 | — | 84.7 |
| Washed with HeptA 1x | 4:1 (25° C.) | 0.3 | 14.9 | 79.6 |
| Washed with HeptA 2x | 4:1 (25° C.) | — | 14.1 | 81.7 |

As can be seen from the examples, it is possible to replace the MCB by the heptanoic acid in such an amount that no more MCB could be detected in the filter cake.

1.-13. (canceled)

14. A process for working up a composition comprising solid 4,4′-dichlorodiphenyl sulfoxide and a solvent, wherein the amount of solvent in the composition is in the range between 5 and 25 wt % based on the total mass of the composition, the process comprising washing the composition using a carboxylic acid until the amount of solvent in the composition is below 1.5 wt % based on the total amount of the composition after washing.

15. The process according to claim 14, wherein the washing is carried out in a filtration apparatus.

16. The process according to claim 15, wherein the amount of carboxylic acid used for washing the composition is at least 0.15 times the total mass of the composition comprising solid 4,4′-dichlorodiphenyl sulfoxide and solvent.

17. The process according to claim 15, wherein the amount of carboxylic acid in the composition after washing is in the range between 6 and 30 wt % based on the total mass of the composition.

18. The process according to claim 15, wherein the composition after washing is mixed with carboxylic acid to obtain a mixture comprising 4,4′-dichlorodiphenyl sulfoxide and carboxylic acid in a weight ratio of 4,4′-dichlorodiphenyl sulfoxide to carboxylic acid of at least 1:2.

19. The process according to claim 14, wherein after washing the composition comprises 4,4′-dichlorodiphenyl sulfoxide and carboxylic acid in a weight ratio of 4,4′-dichlorodiphenyl sulfoxide to carboxylic acid of at least 1:2.

20. The process according to claim 14, wherein a liquid mixture comprising solvent and carboxylic acid is withdrawn from the washing and the mixture is separated into a first stream comprising substantially solvent and a second stream comprising substantially carboxylic acid.

21. The process according to claim 20, wherein the separation into the first stream and second stream is carried out by distillation.

22. The process according to claim 20, wherein the second stream is recycled into the washing.

23. The process according to claim 20, wherein the first stream is recycled into a production process of 4,4′-dichlorodiphenyl sulfoxide.

24. The process according to claim 14, wherein the composition comprising solid 4,4′-dichlorodiphenyl sulfoxide and solvent is a filter cake obtained by filtering a suspension containing 4,4′-dichlorodiphenyl sulfoxide in the solvent.

25. The process according to claim 14, wherein the solvent is chlorobenzene.

26. The process according to claim 14, wherein the carboxylic acid is at least one aliphatic C₆ to C₁₀ carboxylic acid.
